(19) United States
(12) Patent Application Publication
(10) Pub. No.: US 2015/0252407 A1
(43) Pub. Date: Sep. 10, 2015

(54) NANOSTRUCTURE AND METHODS OF NUCLEIC ACID ISOLATION

(71) Applicant: Nvigen, Inc., Sunnyvale, CA (US)

(72) Inventors: Aihua Fu, Sunnyvale, CA (US); Yiguo Shen, Sunnyvale, CA (US)

(73) Assignee: Nvigen, Inc., Sunnyvale, CA (US)

(21) Appl. No.: 14/201,918

(22) Filed: Mar. 9, 2014

(51) Int. Cl. C12Q 1/68; B01J 20/02; B01J 20/22; B01J 20/26; B01J 20/28; B01J 20/06

(52) U.S. Cl. CPC C12Q 1/6806; B01J 20/28009; B01J 20/06; B01J 20/22; B01J 20/261; B01J 20/027

(57) ABSTRACT

A kit comprising a nanostructure comprising at least one core nanoparticle, and a silanization coating on the surface of the core nanoparticle, and a binding buffer comprising a plurality of ingredients at concentration suitable to adjust the concentration of the plurality of ingredients in a solution containing at least one nucleic acid to concentration suitable for binding the nucleic acid through non-hybridization interaction to the nanostructure. A method of using the kit for reversibly binding nucleic acids through non-hybridization based interaction to a nanostructure is also provided.

### FIELD OF THE INVENTION

The present invention generally relates to molecular biology. More specifically, the present application is in the field of nucleic acid isolation.

### BACKGROUND OF THE INVENTION

Nucleic acid analysis plays a major role in the field of diagnostics and bioanalytics in research and development. Before a nucleic acid can be analyzed in a biospecific assay or used for other processes, it often must be isolated or purified from biological samples containing complex mixtures of different components such as proteins. Moreover, nucleic acids are often present in very small concentrations in a biological sample. As a result, nucleic acid presents a special challenge in terms of isolating them from their natural environment. Therefore, new methods of nucleic acid isolation are needed to improve the efficiency and/or sensitivity.

### BRIEF SUMMARY OF THE INVENTION

The present disclosure provides a kit for reversibly binding nucleic acids to a nanostructure. Also provided is a method for reversibly binding nucleic acid through non-hybridization interaction to a nanostructure.

In one aspect, the present disclosure provides a kit comprising a nanostructure and a binding buffer. The nanostructure comprises at least one core nanoparticle and a silanization coating on the surface of the core nanoparticle. The binding buffer comprises a plurality of ingredients at concentration suitable to adjust the concentration of the plurality of ingredients in a solution containing at least one nucleic acid to concentration suitable for binding the nucleic acid through non-hybridization interaction to the nanostructure.

In certain embodiments, the kit comprises a nanostructure comprising at least one core nanoparticle and a silanization coating on the surface of the core nanoparticle, wherein the silanization coating does not include a carboxyl group. In certain embodiment, the core nanoparticle comprises a superparamagnetic iron oxide (SPIO) nanoparticle. In certain embodiment, the silanization coating forms a low density, porous 3-D structure.

In certain embodiment, the kit comprises a nanostructure and a binding buffer. The binding buffer comprises a plurality of ingredients at concentration suitable to adjust the concentration of the plurality of ingredients in a solution containing at least one nucleic acid to concentration suitable for binding the nucleic acid through non-hybridization interaction to the nanostructure, wherein the plurality of ingredients of the solution comprises salt and polyethylene glycol. In some embodiments, the plurality of ingredients of the solution further comprises ingredients selected from the group consisting of acid, base, dNTP, amino acids, sugar, lipid, protein and carbohydrate. In certain embodiments, the polyethylene glycol has a molecular weight of between about 6000 and about 10,000, and wherein the salt is selected from the group consisting of sodium chloride, magnesium chloride, calcium chloride, potassium chloride, lithium chloride, barium chloride and cesium chloride. In certain embodiments, the concentration of the polyethylene glycol suitable for binding the nucleic acid to the nanostructure is between about 5% and about 15% and wherein the concentration of salt suitable for binding the nucleic acid to the nanostructure is between about 0.5 M and about 5.0 M. In certain embodiment, the concentration of the polyethylene glycol suitable for binding the nucleic acid to the nanostructure is about 9.375% and the concentration of salt suitable for binding the nucleic acid to the nanostructure is about 0.625 M. In certain embodiment, the concentration of the polyethylene glycol suitable for binding the nucleic acid to the nanostructure is about 10% and the concentration of salt suitable for binding the nucleic acid to the nanostructure is about 2.0 M. In certain embodiment, the concentration of the polyethylene glycol suitable for binding the nucleic acid to the nanostructure is about 13.3% and the concentration of salt suitable for binding the nucleic acid to the nanostructure is about 1.33 M. In certain embodiment, the concentration of the polyethylene glycol suitable for binding the nucleic acid to the nanostructure is about 15% and the concentration of salt suitable for binding the nucleic acid to the nanostructure is about 1.0 M.

In certain embodiments, the kit further comprises a suitable elution buffer, wherein the elution buffer is capable of releasing the bound nucleic acids from the nanostructure into the elution buffer.

In another aspect, the present disclosure provides a method for reversibly binding at least one nucleic acid through non-hybridization interaction to a nanostructure comprising providing a nanostructure comprising at least one core nanoparticle and a silanization coating on the surface of the core nanoparticle, contacting the nanostructure with a solution containing a first nucleic acid, wherein the concentration of a plurality of ingredients of the solution is adjusted to a concentration suitable for binding the first nucleic acid to the nanostructure, thereby producing a first combination comprising the nanostructure-bound first nucleic acid.

In certain embodiments, the silanization coating does not include carboxyl group. In certain embodiments, the core nanoparticle comprises a superparamagnetic iron oxide (SPIO) nanoparticle. In certain embodiment, the silanization coating forms a low density, porous 3-D structure.

In certain embodiment, the nucleic acid contained in the solution to be used in the method is at sub-nanogram level.

In certain embodiments, the plurality of ingredients of the solution comprises salt and polyethylene glycol. In certain embodiment, the plurality of ingredients of the solution further comprises ingredients selected from the group consisting of acid, base, dNTP, amino acids, sugar, lipid, protein or carbohydrate. In certain embodiment polyethylene glycol in the solution has a molecular weight of between about 6000 and about 10,000, and the salt in the solution is selected from the group consisting of sodium chloride, magnesium chloride, calcium chloride, potassium chloride, lithium chloride, barium chloride and cesium chloride.

In certain embodiments, the method comprises adjusting the concentration of the polyethylene glycol to between about 5% and about 15% and adjusting the concentration of salt to between about 0.5 M and about 5.0 M.

In certain embodiments, the solution containing the first nuclei acid to be used in the method is a cleared lysate. In certain embodiments, the solution containing the first nucleic acid is the reaction product of a PCR amplification. In certain embodiment, the solution containing the first nucleic acid is a biological sample. In certain embodiment, the biological sample is selected from the group consisting of a whole blood, plasma and serum sample.

In certain embodiments, the method further comprising: separating the nanostructure from the first combination; contacting the nanostructure separated from the first combination with the bound nucleic acid in an elution buffer, whereby the nucleic acid bound to the nanostructure is dissociated from the nanostructure; and separating the nanostructure from the elution buffer.

In certain embodiments, the first nucleic acid includes nucleic acid with a size of less than 50, 100, 150, 200, 250, 300, 350, 400, 500, 600, 700, 800, 900, 1 k, 2 k, 3 k, 4 k, 5 k, 10 k or 100 k nucleotides.

In certain embodiments, the solution containing the first nucleic acid further comprises a second nucleic acid of smaller size than the first nucleic acid, and wherein the second nucleic acid of smaller size does not bind to the nanostructure at the concentration of the plurality of ingredients suitable for binding the first nucleic acid to the nanostructure, and the method further comprising: separating the nanostructure-bound first nucleic acid from the first combination; permitting the unbound second nucleic acid of smaller size in the first combination to bind to a second nanostructure, producing a second combination comprising nanostructure-bound second nucleic acid of smaller size; separating the nanostructure-bound second nucleic acid of smaller size from the second combination; contacting the nanostructure-bound second nucleic acid of smaller size separated from the second combination with an elution buffer to release the bound second nucleic acid from the second nanostructure into the elution buffer; and separating the second nanostructure from the elution buffer to provide the second nucleic acid that are substantially free of the first nucleic acid.

In certain embodiments, the solution containing the first nucleic acid further comprises a second nucleic acid of smaller size than the first nucleic acid, and wherein the second nucleic acid of smaller size does not bind to the nanostructure at the concentration of the plurality of ingredients suitable for binding the first nucleic acid to the nanostructure, and the method further comprising: separating the nanostructure-bound first nucleic acid from the first combination; permitting the unbound second nucleic acid of smaller size in the first combination to bind to a second nanostructure, producing a second combination comprising nanostructure-bound second nucleic acid of smaller size; separating the nanostructure-bound second nucleic acid of smaller size from the second combination; contacting the nanostructure-bound second nucleic acid of smaller size separated from the second combination with an elution buffer to release the bound second nucleic acid from the second nanostructure into the elution buffer; and separating the second nanostructure from the elution buffer to provide the second nucleic acid that are substantially free of the first nucleic acid.

In yet another aspect, the present disclosure provides a composition for reversibly binding nucleic acids through non-hybridization interaction comprising at least one core nanoparticle, and a silanization coating on the surface of the core nanoparticle, wherein the silanization coating does not include carboxyl group.

### BRIEF DESCRIPTION OF THE FIGURES

FIG. 1. Selective isolation of small size nucleic acids using nanostructure. DNA ladder were isolated using nanostructure with size selection buffer. FIG. 1 illustrates that DNA of different size were selectively isolated using different size selection buffer. Notice that using size selection buffer II and III can isolate DNA having size smaller than 50 bp.

FIG. 2. Isolation of salmon sperm DNA using nanostructure. Salman sperm DNA were isolated using nanostructure (Mag) or commercially available nanoparticles (Ampure). The bound DNA were resolved on 3% agarose gel. FIG. 2 illustrates that the yield of DNA capture using the nanostructure is comparable to the commercially available nanoparticle (Ampure).

FIG. 3. The recovery rate of DNA isolation using nanostructure. Genomic DNA from OC1-LY8 cells and salmon sperm DNA were isolated using nanostructure or commercially available nanoparticles (Ampure). DNA captured by nanostructure were eluted and quantified using Picogreen fluorescent reagent (Life Technologies). FIG. 3 illustrates that the capturing efficiency of nanostructure (dark) and commercially available nanoparticles (light) are comparable.

FIG. 4. Higher DNA binding capacity using nanostructure. Salmon sperm DNA were isolated using nanostructure (Magvigen) or commercially available nanoparticles (Ampure) and analyzed on 3% agrose gel. FIG. 4 illustrates that using nanostructure yield a significant higher binding capacity than commercially available nanoparticles under the saturation conditions.

FIG. 5. The recovery rate of PCR product clean up using nanostructure. Incubate 20 ul of Magvigen nanoparticles with Different size (100, 250, 500 or 1,000 ng) DNA were isolated using nanostructure. The bounded DNA were eluted in TE and quantified using Picogreen fluorescent reagent. FIG. 5 illustrates that the recovery rate is above 75% percent and remains stable within all DNA concentration range.

FIG. 6. Detection of trace amount of DNA using nanostructure. A DNA template of 82 bp was spiked into human blood sample, a biotin-labeled primer was used to specifically target this 82 bp template in blood sample. The DNA template in the blood sample were isolated using nanostructure coated with Streptavidin (MagVigen). The isolated DNA template was further amplified by PCR and analyzed on 3% agrose gel. FIG. 6 shows that Streptavidin coated nanostructure can detect DNA to as low as 50 pg/ml blood, and there is no-unspecific binding.

FIG. 7. Isolation of plasma DNA using nanostructure. DNA ladder added into plasma were isolated using nanostrucutre. The isolated DNA were resolved on agarose gel.

FIG. 8. Comparison between non-carboxyl coated and carboxyl coated nanostructure. DNA ladder were isolated using non-carboxyl or carboxyl coated nanostructure. The bounded DNA were eluted in TE and quantified using Picogreen fluorescent reagent. FIG. 8 illustrates that the yield of non-carboxyl coated nanostructure is higher than the carboxyl coated nanostructure.

FIG. 9. Comparison between non-carboxyl coated and carboxyl coated nanostructure. DNA ladders were isolated using non-carboxyl or carboxyl coated nanostructure with size selection buffer. The bound DNA were eluted and resolved on agarose gel or quantified using picogreen fluorescent reagent. FIG. 9 illustrates that the yield of non-carboxyl coated nanostructure is higher than the carboxyl coated nanostructure.

FIG. 10. Selective isolation of nucleic acid using nanostructure. DNA ladder were isolated using nanostructure and size selection buffers. FIG. 10 illustrates that using different size selection buffer resulted in selective binding of DNA of different sizes to the nanostructure.

FIG. 11. Isolation of a medium size range of DNA using nanostructure. Salmon sperm DNA (left panel) or DNA Ladder (right panel) were isolated using nanostructure following two steps extraction protocol. FIG. 11 illustrates that the 300 bp-600 bp DNA were isolated.

### DETAILED DESCRIPTION OF THE INVENTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present application will be limited only by the appended claims. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, solid state chemistry, inorganic chemistry, organic chemistry, physical chemistry, analytical chemistry, materials chemistry, biochemistry, biology, molecular biology, recombinant DNA techniques, pharmacology, imaging, and the like, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” series (Academic Press, Inc., 1955-2014); “Current Protocols in Molecular Biology” (F. M. Ausubel et al., eds., 1987, and periodic updates); “PCR: The Polymerase Chain Reaction”, (Mullis et al., eds., 1994). Primers, polynucleotides and polypeptides employed in the present application can be generated using standard techniques known in the art.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

The following embodiments are put forth so as to provide those of ordinary skill in the art with a complete disclosure and description of how to perform the methods and use the nanostructure disclosed and claimed herein. Efforts have been made to ensure accuracy with respect to numbers (e.g., amounts, temperature, etc.), but some errors and deviations should be accounted for.

It must be noted that, as used in the specification and the appended claims, the singular forms “a,” “an,” and “the” include plural forms of the same unless the context clearly dictates otherwise. Thus, for example, reference to “a compound” includes a plurality of compounds. In this specification and in the claims that follow, reference will be made to a number of terms that shall be defined to have the following meanings unless a contrary intention is apparent.

In one aspect, the present disclosure provides a kit comprising a nanostructure and a binding buffer. The nanostructure comprises at least one core nanoparticle and a silanization coating on the surface of the core nanoparticle. The binding buffer comprises a plurality of ingredients at concentration suitable to adjust the concentration of the plurality of ingredients in a solution containing at least one nucleic acid to concentration suitable for binding the nucleic acid through non-hybridization interaction to the nanostructure.

The term “nanostructure” as used herein, refers to a particle having a diameter ranging from about 1 nm to about 1500 nm (e.g. from 1 nm to 1200 nm, from 1 nm to 1000 nm, from 1 nm to 800 nm, from 1 nm to 500 nm, from 1 nm to 400 nm, etc.). Such a nanostructure has been described in US Patent Application Serial No US 20100008862 A1, PCT Application Serial No WO2013112643, which are incorporated in whole and in part to the present application. In certain embodiment, the nanostructure comprises a single particle or a cluster of particles. In certain embodiments, the nanostructure comprises a core nanoparticle and a coating. The core nanoparticle can be a single or a cluster of particles. The coating can be any coating known in the art, for example, a polymer coating such as polyethylene glycol, silane, and polysaachrides (e.g. dextran and its derivatives).

In some embodiments, the nanostructures provided herein contain a magnetic material. Suitable magnetic materials include, for example, ferrimagnetic or ferromagnetic materials (e.g., iron, nickel, cobalt, some alloys of rare earth metals, and some naturally occurring minerals such as lodestone), paramagnetic materials (such as platinum, aluminum), and superparamagnetic materials (e.g., superparamagnetic iron oxide or SPIO, and SPIO doped with other elements such as Mg, Cd, Ag, Au, Mn, Co, Ni, Zn, Ca.).

The magnetic material has magnetic property which allows the nanostructure to be pulled or attracted to a magnet or in a magnetic field. Magnetic property can facilitate manipulation (e.g., separation, purification, or enrichment) of the nanostructures using magnetic interaction. The magnetic nanostructures can be attracted to or magnetically guided to an intended site when subject to an applied magnetic field, for example a magnetic field from high-filed and/or high-gradient magnets. For example, a magnet (e.g., magnetic grid) can be placed in the proximity of the nanostructures so as to attract the magnetic nanostructures.

Any nanostructures having a magnetic property known in the art can be used. In certain embodiments, the nanostructure provided herein comprises a magnetic nanoparticle which comprises a magnetic material. For example, the magnetic nanoparticle of the nanostructure can be a superparamagnetic iron oxide (SPIO) nanoparticle. The SPIO nanoparticle is an iron oxide nanoparticle, either magnemite (γ-Fe₂O₃) or magnetite (Fe₃O₄), or nanoparticles composed of both phases.

The SPIO nanoparticle can be made using one or more methods. For example, SPIO nanoparticles can be synthesized with a suitable method and dispersed as a colloidal solution in organic solvents or water. Methods to synthesize the SPIO nanoparticles are known in the art (see, for example, Morteza Mahmoudi et al, Superparamagnetic Iron Oxide Nanoparticles: Synthesis, Surface Engineering, Cytotoxicity and Biomedical Applications, published by Nova Science Pub Inc, 2011). In one embodiment, the SPIO nanoparticles can be made through wet chemical synthesis methods which involve co-precipitation of Fe and Fe salts in the presence of an alkaline medium. During the synthesis, nitrogen may be introduced to control oxidation, surfactants and suitable polymers may be added to inhibit agglomeration or control particle size, and/or emulsions (such as water-in-oil microemulsions) may be used to modulate the physical properties of the SPIO nanoparticle (see, for example, Jonathan W. Gunn, The preparation and characterization of superparamagnetic nanoparticles for biomedical imaging and therapeutic application, published by ProQuest, 2008). In another embodiment, the SPIO nanoparticles can be generated by thermal decomposition of iron pentacarbonyl, alone or in combination with transition metal carbonyls, optionally in the presence of one or more surfactants (e.g., lauric acid and oleic acid) and/or oxidatants (e.g., trimethylamine-N-oxide), and in a suitable solvent (e.g., dioctyl ether or hexadecane) (see, for example, US patent application 20060093555). In another embodiment, the SPIO nanoparticles can also be made through gas deposition methods, which involves laser vaporization of iron in a helium atmosphere containing different concentrations of oxygen (see, Miller J. S. et al., Magnetism: Nanosized magnetic materials, published by Wiley-VCH, 2002). In certain embodiments, the SPIO nanoparticles are those disclosed in US patent application US20100008862.

In certain embodiments, the nanostructure can further comprise a non-SPIO nanoparticle. The non-SPIO nanoparticles include, for example, metallic nanoparticles (e.g., gold or silver nanoparticles (see, e.g., Hiroki Hiramatsu, F.E.O., Chemistry of Materials 16, 2509-2511 (2004)), semiconductor nanoparticles (e.g., quantum dots with individual or multiple components such as CdSe/ZnS (see, e.g., M. Bruchez, et al, science 281, 2013-2016 (1998))), doped heavy metal free quantum dots (see, e.g., Narayan Pradhan et al, J. Am. chem. Soc. 129, 3339-3347 (2007)) or other semiconductor quantum dots); polymeric nanoparticles (e.g., particles made of one or a combination of PLGA (poly(lactic-co-glycolic acid) (see, e.g., Minsoung Rhee et al, Adv. Mater. 23, H79-H83 (2011)), PCL (polycaprolactone) (see, e.g., Marianne Labet et al, Chem. Soc. Rev. 38, 3484-3504 (2009)), PEG (poly ethylene glycol) or other polymers); siliceous nanoparticles; and non-SPIO magnetic nanoparticles (e.g., MnFe2O4 (see, e.g., Jae-Hyun Lee et al, Nature Medicine 13, 95-99 (2006)), synthetic antiferromagnetic nanoparticles (SAF) (see, e.g., A. Fu et al, Angew. Chem. Int. Ed. 48, 1620-1624 (2009)), and other types of magnetic nanoparticles). In certain embodiments, the non-SIPO nanoparticle is a colored nanoparticle, for example, a semiconductor nanoparticle such as a quantum dot.

The non-SPIO nanoparticles can be prepared or synthesized using suitable methods known in the art, such as for example, sol-gel synthesis method, water-in-oil micro-emulsion method, gas deposition method and so on. For example, gold nanoparticles can be made by reduction of chloroaurate solutions (e.g., HAuCl₄) by a reducing agent such as citrate, or acetone dicarboxulate. For another example, CdS semiconductor nanoparticle can be prepared from Cd(ClO₄)₂ and Na₂S on the surface of silica particles. For another example, II-VI semiconductor nanoparticles can be synthesized based on pyrolysis of organometallic reagents such as dimethyl cadmium and trioctylphosphine selenide, after injection into a hot coordinating solvent (see, e.g., Gunter Schmid, Nanoparticles: From Theory to Application, published by John Wiley & Sons, 2011). Doped heavy metal free quantum dots, for example Mn-doped ZnSe quantum dots can be prepared using nucleation-doping strategy, in which small-sized MnSe nanoclusters are formed as the core and ZnSe layers are overcoated on the core under high temperatures. For another example, polymeric nanoparticles can be prepared by emulsifying a polymer in a two-phase solvent system, inducing nanosized polymer droplets by sonication or homogenization, and evaporating the organic solvent to obtain the nanoparticles. For another example, siliceous nanoparticles can be prepared by sol-gel synthesis, in which silicon alkoxide precursors (e.g., TMOS or TEOS) are hydrolyzed in a mixture of water and ethanol in the presence of an acid or a base catalyst, the hydrolyzed monomers are condensed with vigorous stirring and the resulting silica nanoparticles can be collected. For another example, SAFs, a non-SPIO magnetic nanoparticle, can be prepared by depositing a ferromagenetic layer on each of the two sides of a nonmagnetic space layer (e.g., ruthenium metal), along with a chemical etchable copper release layer and protective tantalum surface layers, using ion-bean deposition in a high vacuum, and the SAF nanoparticle can be released after removing the protective layer and selective etching of copper.

In certain embodiments, the nanostructure comprises a combination of SPIO and non-SPIO nanoparticles.

The size of the nanoparticles ranges from 1 nm to 900 nm in size (preferable 100-800 nm, 100-700 nm, 100-600 nm, 100-500 nm, 100-400 nm, 100-300 nm, 100-200 nm, 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 100 nm, 200 nm, 300 nm, 400 nm, 500 nm, 600 nm, 700 nm, 800 nm, 900 nm in size). The size of nanoparticles can be controlled by selecting appropriate synthesis methods and/or systems. For example, to control the size of nanoparticles, synthesis of nanoparticles can be carried out in a polar solvent which provides ionic species that can adsorb on the surface of the nanoparticles, thereby providing electrostatic effect and particle-particle repulsive force to help stabilize the nanoparticles and inhibit the growth of the nanoparticles. For another example, nanoparticles can be synthesized in a micro-heterogeneous system that allows compartmentalization of nanoparticles in constrained cavities or domains. Such a micro-heterogeneous system may include, liquid crystals, mono and multilayers, direct micelles, reversed micelles, microemulsions and vesicles. To obtain nanoparticles within a desired size range, the synthesis conditions may be properly controlled or varied to provide for, e.g., a desired solution concentration or a desired cavity range (a detailed review can be found at, e.g., Vincenzo Liveri, Controlled synthesis of nanoparticles in microheterogeneous systems, Published by Springer, 2006).

The shape of the nanoparticles can be spherical, cubic, rod shaped (see, e.g., A. Fu et al, Nano Letters, 7, 179-182 (2007)), tetrapo-shaped (see, e.g., L. Manna et al, Nature Materials, 2, 382-385 (2003)), pyramidal, multi-armed, nanotube, nanowire, nanofiber, nanoplate, or any other suitable shapes. Methods are known in the art to control the shape of the nanoparticles during the preparation (see, e.g., Waseda Y. et al., Morphology control of materials and nanoparticles: advanced materials processing and characterization, published by Springer, 2004). For example, when the nanoparticles are prepared by the bottom-up process (i.e. from molecule to nanoparticle), a shape controller which adsorbs strongly to a specific crystal plane may be added to control the growth rate of the particle.

A single nanostructure may comprise a single nanoparticle or a plurality or a cluster of mini-nanoparticles (A. Fu et al, J. Am. chem. Soc. 126, 10832-10833 (2004), J. Ge et al, Angew. Chem. Int. Ed. 46, 4342-4345 (2007), Zhenda Lu et al, Nano Letters 11, 3404-3412 (2011).). The mini-nanoparticles can be homogeneous (e.g., made of the same composition/materials or having same size) or heterogeneous (e.g., made of different compositions/materials or having different sizes). A cluster of homogeneous mini-nanoparticles refers to a pool of particles having substantially the same features or characteristics or consisting of substantially the same materials. A cluster of heterogeneous mini-nanoparticles refers to a pool of particles having different features or characteristics or consisting of substantially different materials. For example, a heterogeneous mini-nanoparticle may comprise a quantum dot in the center and a discrete number of gold (Au) nanocrystals attached to the quantum dot. When the nanoparticles are associated with a coating (as described below), different nanoparticles in a heterogeneous nanoparticle pool do not need to associate with each other at first, but rather, they could be individually and separately associated with the coating.

In certain embodiments, a nanostructure disclosed comprises a plurality of nanoparticles. For example, the nanostructure contains 2, 3, 4, 5, 6, 7, 8, 9, 10, 11, 12, 13, 14, 15, 16, 17, 18, 19, 20, 25, 30, 35, 40, 50, 60, 70, 80, 90, 100, 100 s or 1000 s nanoparticles.

In certain embodiments, the nanostructure provided herein further comprises a coating. At least one core nanoparticle can be embedded in or coated with the coating. Any suitable coatings known in the art can be used, for example, a polymer coating and a non-polymer coating. The coating interacts with the core nanoparticles through 1) intra-molecular interaction such as covalent bonds (e.g., Sigma bond, Pi bond, Delta bond, Double bond, Triple bond, Quadruple bond, Quintuple bond, Sextuple bond, 3c-2e, 3c-4e, 4c-2e, Agostic bond, Bent bond, Dipolar bond, Pi backbond, Conjugation, Hyperconjugation, Aromaticity, Hapticity, and Antibonding), metallic bonds (e.g., chelating interactions with the metal atom in the core nanoparticle), or ionic bonding (cation π-bond and salt bond), and 2) inter-molecular interaction such as hydrogen bond (e.g., Dihydrogen bond, Dihydrogen complex, Low-barrier hydrogen bond, Symmetric hydrogen bond) and non covalent bonds (e.g., hydrophobic, hydrophilic, charge-charge, or π-stacking interactions, van der Waals force, London dispersion force, Mechanical bond, Halogen bond, Aurophilicity, Intercalation, Stacking, Entropic force, and chemical polarity).

In certain embodiments, the nanostructure comprises a silanization coating on the surface of the nanoparticle. In an embodiment, the silanization coating is a coating including silane and/or silane-like molecules (or the reaction products of those molecules with the surface) onto the surface of the SPIO nanoparticles. The coating can be amorphous. The thickness of the coating can be controlled so that coated SPIO nanoparticles can be created for particular applications. In an embodiment, the silanization coating is made by cross-linking of trimethoxyl silanes with appropriate functional groups, such as a mercapto group, an amino group, a mercapto/amino group, a carboxyl group, a phosphonate group, an alkyl group, a polyethylene oxide group (PEG), and combinations thereof.

In an embodiment, the silanization coating can be about 1 to 5 nm thick. In an embodiment, the silanization coating can be about 1 to 10 nm thick. In an embodiment, the silanization coating can be about 1 to 20 nm thick. In an embodiment, the silanization coating can be about 1 to 30 nm thick. In an embodiment, the silanization coating can be about 1 to 40 nm thick. In an embodiment, the silanization coating can be about 1 to 50 nm thick. In an embodiment, the silanization coating can be about 1 to 60 nm thick. In an embodiment, the silanization coating can be about 1 to 100 nm thick. In an embodiment, the silanization coating can be about 1 to 500 nm thick. In an embodiment, the silanization coating can be about 1 to 1000 nm thick. In an embodiment, a silanization thickness of 2-3 nm is enough to provide a robust coating that will keep nanoparticle stable (e.g., no aggregates or sediments formed) inside physiological buffer (e.g., phosphate buffered saline with a pH of about 7.3) for greater than 6 months, greater than a year, 3-5 years, or longer. Thicker silanization coating can also be rationally controlled by adding a larger amount of trimethoxyl silane reagents or using sodium silicate.

In certain embodiments, the coating comprises a low density, porous 3-D structure, as disclosed in U.S. Prov. Appl. 61/589,777 and U.S. patent application Ser. No. 12/460,007 (all references cited in the present disclosure are incorporated herein in their entirety).

The low density, porous 3-D structure refers to a structure with density much lower (e.g., 10 s times, 20 s times, 30 s times, 50 s times, 70 s times, 100 s times) than existing mesoporous nanoparticles (e.g., mesoporous nanoparticles having a pore size ranging from 2 nm to 50 nm). (A. Vincent, et. al., J. Phys. Chem. C, 2007, 111, 8291-8298. J. E. Lee, et. al, J. Am. Chem. Soc, 2010, 132, 552-557. Y.-S. Lin, et. al, J. Am. Chem. Soc, 2011, 133, 20444-20457. Z. Lu, Angew. Chem. Int. Ed., 2010, 49, 1862-1866.)

In certain embodiments, the low density, porous 3-D structure refers to a structure having a density of <1.0 g/cc (e.g., <100 mg/cc, <10 mg/cc, <5 mg/cc, <1 mg/cc, <0.5 mg/cc, <0.4 mg/cc, <0.3 mg/cc, <0.2 mg/cc, or <0.1 mg/cc) (for example, from 0.01 mg/cc to 10 mg/cc, from 0.01 mg/cc to 8 mg/cc, from 0.01 mg/cc to 5 mg/cc, from 0.01 mg/cc to 3 mg/cc, from 0.01 mg/cc to 1 mg/cc, from 0.01 mg/cc to 1 mg/cc, from 0.01 mg/cc to 0.8 mg/cc, from 0.01 mg/cc to 0.5 mg/cc, from 0.01 mg/cc to 0.3 mg/cc, from 0.01 mg/cc to 1000 mg/cc, from 0.01 mg/cc to 915 mg/cc, from 0.01 mg/cc to 900 mg/cc, from 0.01 mg/cc to 800 mg/cc, from 0.01 mg/cc to 700 mg/cc, from 0.01 mg/cc to 600 mg/cc, from 0.01 mg/cc to 500 mg/cc, from 0.1 mg/cc to 800 mg/cc, from 0.1 mg/cc to 700 mg/cc, from 0.1 mg/cc to 1000 mg/cc, from 1 mg/cc to 1000 mg/cc, from 5 mg/cc to 1000 mg/cc, from 10 mg/cc to 1000 mg/cc, from 20 mg/cc to 1000 mg/cc, from 30 mg/cc to 1000 mg/cc, from 30 mg/cc to 1000 mg/cc, from 30 mg/cc to 900 mg/cc, from 30 mg/cc to 800 mg/cc, or from 30 mg/cc to 700 mg/cc).

The density of 3-D structure can be determined using various methods known in the art (see, e.g., Lowell, S. et al, Characterization of porous solids and powders: surface area, pore size and density, published by Springer, 2004). Exemplary methods include, Brunauer Emmett Teller (BET) method and helium pycnometry (see, e.g., Varadan V. K. et al., Nanoscience and Nanotechnology in Engineering, published by World Scientific, 2010). Briefly, in BET method, dry powders of the testing 3-D structure is placed in a testing chamber to which helium and nitrogen gas are fed, and the change in temperature is recorded and the results are analyzed and extrapolated to calculate the density of the testing sample. In helium pycnometry method, dry powders of the testing 3-D structure are filled with helium, and the helium pressure produced by a variation of volume is studied to provide for the density. The measured density based on the dry power samples does not reflect the real density of the 3-D structure because of the ultralow density of the 3-D structure, the framework easily collapses during the drying process, hence providing much smaller numbers in the porosity measurement than when the 3-D structure is fully extended, for example, like when the 3-D structure is fully extended in a buffer solution. In certain embodiments, the density of the 3-D structure can be determined using the dry mass of the 3-D structure divided by the total volume of such 3-D structure in an aqueous solution. For example, dry mass of the core particles with and without the 3-D structure can be determined respectively, and the difference between the two would be the total mass of the 3-D structure. Similarly, the volume of a core particle with and without the 3-D structure in an aqueous solution can be determined respectively, and the difference between the two would be the volume of the 3-D structure on the core particle in an aqueous solution.

In certain embodiments, the porous nanostructure can be dispersed as multiple large nanoparticles coated with the 3-D structure in an aqueous solution, in such case, the total volume of the 3-D structure can be calculated as the average volume of the 3-D structure for an individual large nanoparticle multiplied with the number of the large nanoparticles. For each individual large nanoparticle, the size (e.g., radius) of the particle with 3-D structure can be determined with Dynamic Light Scattering (DLS) techniques, and the size (e.g., radius) of the particle core without the 3-D structure can be determined under Transmission Electron Microscope (TEM), as the 3-D structure is substantially invisible under TEM. Accordingly, the volume of the 3-D structure on an individual large nanoparticle can be obtained by subtracting the volume of the particle without 3-D structure from the volume of the particle with the 3-D structure.

The number of large nanoparticles for a given core mass can be calculated using any suitable methods. For example, an individual large nanoparticle may be composed of a plurality of small nanoparticles which are visible under TEM. In such case, the average size and volume of a small nanoparticle can be determined based on measurements under TEM, and the average mass of a small nanoparticle can be determined by multiplying the known density of the core material with the volume of the small particle. By dividing the core mass with the average mass of a small nanoparticle, the total number of small nanoparticles can be estimated. For an individual large nanoparticle, the average number of small nanoparticles in it can be determined under TEM. Accordingly, the number of large nanoparticles for a given core mass can be estimated by dividing the total number of small nanoparticles with the average number of small nanoparticels in an individual large nanoparticle. Alternatively, the low density, porous 3-D structure refers to a structure having 40%-99.9% (preferably 50% to 99.9%) of empty space or pores in the structure, where 80% of the pores having size of 1 nm to 500 nm in pore radius.

The porosity of the 3-D structure can be characterized by the Gas/Vapor adsorption method. In this technique, usually nitrogen, at its boiling point, is adsorbed on the solid sample. The amount of gas adsorbed at a particular partial pressure could be used to calculate the specific surface area of the material through the Brunauer, Emmit and Teller (BET) nitrogen adsorption/desorption equation. The pore sizes are calculated by the Kelvin equation or the modified Kelvin equation, the BJH equation (see, e.g., D. Niu et al, J. Am. chem. Soc. 132, 15144-15147 (2010)). The porosity of the 3-D structure can also be characterized by mercury porosimetry (see, e.g., Varadan V. K. et al, supra). Briefly, gas is evacuated from the 3-D structure, and then the structure is immersed in mercury. As mercury is non-wetting at room temperature, an external pressure is applied to gradually force mercury into the sample. By monitoring the incremental volume of mercury intruded for each applied pressure, the pore size can be calculated based on the Washburn equation.

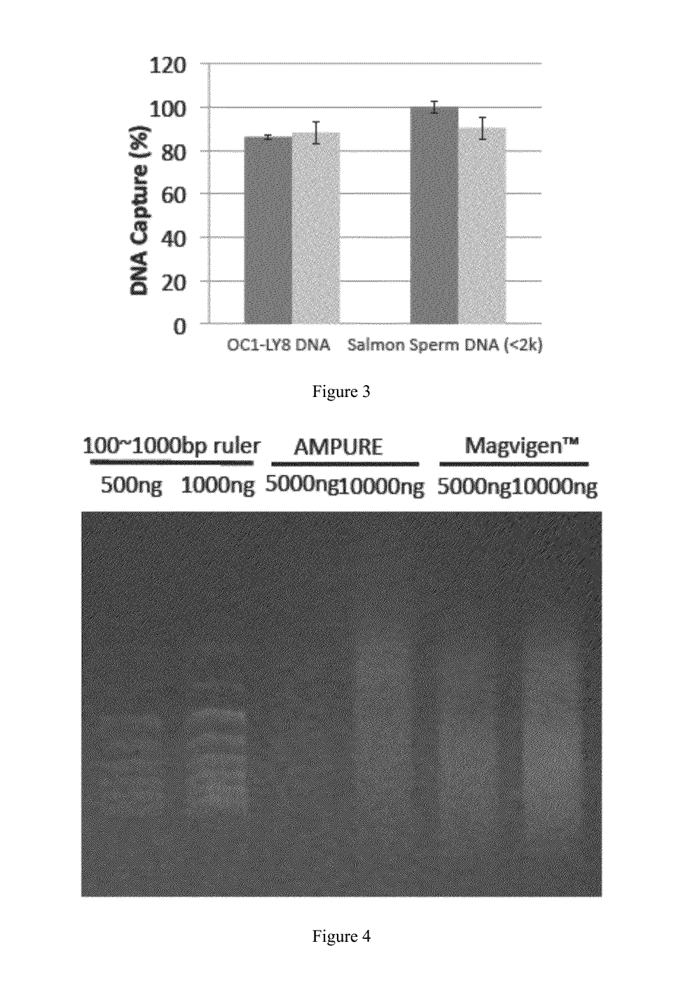

Alternatively, the low density, porous 3-D structure refers to a structure that has a material property, that is, the porous structure (except to the core nanoparticle or core nanoparticles) could not be obviously observed or substantially transparent under transmission electron microscope, for example, even when the feature size of the 3-D structure is in the 10 s or 100 s nanometer range. The term “obviously observed” or “substantially transparent” as used herein means that, the thickness of the 3-D structure can be readily estimated or determined based on the image of the 3-D structure under TEM. The nanostructure (e.g., nanoparticles coated with or embedded in/on a low density porous 3-D structure) can be observed or measured by ways known in the art. For example, the size (e.g., radius) of the nanostructure with the 3-D structure can be measured using DLS methods, and the size (e.g., radius) of the core particle without the 3-D structure can be measured under TEM. In certain embodiments, the thickness of the 3-D structure is measured as 10 s, 100 s nanometer range by DLS, but cannot be readily determined under TEM. For example, when the nanostructures provided herein are observed under Transmission Electron Microscope (TEM), the nanoparticles can be identified, however, the low density porous 3-D structure can not be obviously observed, or is almost transparent (e.g., see FIGS. 2 and 3). This distinguishes the nanostructures provided herein from those reported in the art that comprise nanoparticles coated with crosslinked and size tunable 3-D structure, including the mesoporous silica nanoparticles or coating (see, e.g., J. Kim, et. al, J. Am. Chem. Soc, 2006, 128, 688-689; J. Kim, et. al, Angew. Chem. Int. Ed., 2008, 47, 8438-8441). This feature also indicates that the low density porous 3-D structure provided herein has a much lower density and/or is highly porous in comparison to other coated nanoparticles known in the art. The porosity of the 3-D structure can be further evaluated by the capacity to load different molecules (see, e.g., Wang L. et al, Nano Research 1, 99-115 (2008)). As the 3-D structure provided herein has a low density, it is envisaged that more payload can be associated with the 3-D structure than with other coated nanoparticles. For example, when 3-D structure is loaded with organic fluorophores such as Rhodamin, over 105 Rhodamin molecules can be loaded to 3-D structure of one nanoparticle.

In certain embodiments, the low density structure refers to a structure capable of absorbing or carrying a fluorescent payload whose fluorescence intensity is at least 100 fold of that of the free fluorescent molecule (e.g., at least 150 fold, 200 fold, 250 fold, 300 fold, 350 fold, 400 fold, 450 fold, 500 fold, 550 fold or 600 fold). The fluorescence intensity of a loaded nanoparticle can be quantified under the same excitation and emission wave lengths as that of the fluorescent molecules. The fluorescence intensity of the loaded low density structure indicates the payload of the fluorescent molecule, and also indirectly reflects the porosity of the low density structure.

In certain embodiments, the low density, porous 3-D structure is made of silane-containing or silane-like molecules (e.g., silanes, organosilanes, alkoxysilanes, silicates and derivatives thereof).

In certain embodiments, the silane-containing molecule comprises an organosilane, which is also known as silane coupling agent. Organosilane has a general formula of RₓSiY(₄₋ₓ), wherein R group is an alkyl, aryl or organo functional group. Y group is a methoxy, ethoxy or acetoxy group, x is 1, 2 or 3. The R group could render a specific function such as to associate the organosilane molecule with the surface of the core nanoparticle or other payloads through covalent or non-covalent interactions. The Y group is hydro lysable and capable of forming a siloxane bond to crosslink with another organosilane molecule. Exemplary R groups include, without limitation, disulphidealkyl, aminoalkyl, mercaptoalkyl, vinylalkyl, epoxyalkyl, and methacrylalkyl, carboxylalkyl groups. The alkyl group in an R group can be methylene, ethylene, propylene, and etc. Exemplary Y groups include, without limitation, alkoxyl such as OCH₃, OC₂H₅, and OC₂H₄OCH₃. For example, the organosilane can be amino-propyl-trimethoxysilane, mercapto-propyl-trimethoxysilane, carboxyl-propyl-trimethoxysilane, amino-propyl-triethoxysilane, mercapto-propyl-triethoxysilane, carboxyl-propyl-triethoxysilane, Bis-[3-(triethoxysilyl) propyl]-tetrasulfide, Bis-[3-(triethoxysilyl) propyl]-disulfide, aminopropyltriethoxysilane, N-2-(aminoethyl)-3-amino propyltrimethoxysilane, Vinyltrimethoxysilane, Vinyl-tris(2-methoxyethoxy) silane, 3-methacryloxypropyltrimethoxy silane, 2-(3,4-epoxycyclohexy)-ethyl trimethoxysilane, 3-glycidoxy-propyltriethoxysilane, 3-isocyanatopropyltriethoxysilane, and 3-cyanatopropyltriethoxy silane.

In certain embodiments, the silanization coating contains one or more functional groups within in or on the surface of the coating. The functional groups may be introduced during the formation of the coating during the cross-linking process, for example, by adding silicon-containing compounds containing such functional groups during the cross-linking, in particular, during the ending stage of the cross-linking process. The functional groups may also be introduced after the formation of the cross-linking product, for example, by introducing functional groups to the surface of the cross-linking product by chemical modification. In certain embodiments, the functional groups are inherent in the coating.

The functional groups serve as linkage between the nanosructure and payioads (e.g., nucleic acids to be captured). Examples of the functional groups include, but are not limited to amino, mercapto, carboxyl, phosphonate, biotin, streptavidin, avidin, hydroxyl, aikyl or other hydrophobic molecules, polyethylene glycol or other liydrophilic molecules, and photo cleavable, thermo cleavable or pH responsive linkers.

In certain embodiment, the silanization coating does not contain a carboxyl functional group. As illustrated in Example 6, the nanostructure comprising a silanization coating that does not contain a carboxyl group demonstrates a higher binding efficiency as compared to the nanostructure comprising a carboxyl coating.

In certain embodiment, the kit comprises a nanostructure and a binding buffer. The binding buffer comprises a plurality of ingredients at concentration suitable to adjust the concentration of the plurality of ingredients in a solution containing at least one nucleic acid to concentration suitable for binding the nucleic acid through non-hybridization interaction to the nanostructure, wherein the plurality of ingredients of the solution comprises salt and polyethylene glycol.

The term “salt” as used herein, refers to a compound produced by the interaction of an acid and a base. Exemplary salts include, but are not limited to, sodium chloride (table salt), sodium iodide, sodium bromide, lithium bromide, lithium iodide, potassium phosphate, sodium bicarbonate, and the like. In water and other aqueous solutions, salts typically dissociate into an “anion” or negatively charged subcomponent, and a “cation” or positively charge subcomponent. For example, when sodium chloride (NaCl) is dissolved in water, it dissociates into a sodium cation (Na⁺) and a chloride anion (Cl⁻). Exemplary salts are discussed, e.g., in Waser, Jurg, Quantitative Chemistry, A Laboratory Text, W. A. Benjamin, Inc., New York, page 160, (1966).

The term “non-hybridization interaction” used herein refers to the process of establishing a non-covalent, non-sequence-specific interaction between nucleic acid molecules and nanostructure. Through a non-hybridization interaction, binding of different nucleic acid molecules to nanostructure does not require an interaction between two or more complementary strands of nucleic acids or based on the hydrogen bonding between A and T or U, or G and C.

The term “nucleic acid” as used herein, refers to a polymer of ribonucleosides or deoxyribonucleosides typically comprising phosphodiester linkages between subunits. Other linkages between subunits include, but are not limited to, methylphosphonate, phosphorothioate, and peptide linkages. Such nucleic acids include, but are not limited to, genomic DNA, cDNA, single strand DNA, hnRNA, mRNA, rRNA, tRNA, fragmented nucleic acid, nucleic acid obtained from subcellular organelles such as mitochondria or chloroplasts, and nucleic acid obtained from microorganisms or DNA or RNA viruses that may be present on or in a biological sample.

A “solution containing nucleic acid” can be any aqueous solution, such as a solution containing DNA, RNA and/or PNAs. Such a solution can also contain other components, such as other biomolecules, inorganic compounds and organic compounds. The solution can contain DNA which is the reaction product of PCR amplification.

In certain embodiment, the solution is a biological sample. The term “biological sample” is used in a broad sense and is intended to include a variety of biological sources or solutions that contain nucleic acids. Such sources include, without limitation, whole tissues, including biopsy materials and aspirates; in vitro cultured cells, including primary and secondary cells, transformed cell lines, and tissue and cellular explants; whole blood, red blood cells, white blood cells, and lymph; body fluids such as urine, sputum, semen, saliva, secretions, eye washes and aspirates, lung washes, cerebrospinal fluid, abscess fluid, and aspirates. Included in this definition of “biological sample” are samples processed from biological sources, including but not limited to cell lysates and nucleic acid-containing extracts, or serum/plasma samples. A “cell lysate”, as used herein, is a solution containing cells which contain cloned DNA and genomic DNA and whose cell membranes have been disrupted, with the result that the contents of the cell, including the DNA contained therein, are in the solution. Any organism containing nucleic acid may be a source of a biological sample, including, but not limited to, any eukaryotes, eubacteria, archaebacteria, or virus. Fungal and plant tissues, such as leaves, roots, stems, and caps, are also within the scope of the present invention. Microorganisms and viruses that may be present on or in a biological sample are within the scope of the invention.

The solution containing nucleic acid may also be an agarose solution. For example, a mixture of DNA is separated, according to methods known to one skilled in the art, such as by electrophoresis on an agarose gel. A plug of agarose containing DNA of interest can be excised from the gel and added to 1-10 volumes of 0.5×SSC (0.75M NaCl, 0.0075M Sodium Citrate, pH 7.0) preferably 4 volumes. The mixture is then melted at a temperature of from about 60 to 80 minutes, to create an agarose solution containing DNA.

In some embodiments, the plurality of ingredients of the solution further comprises ingredients selected from the group consisting of acid, base, dNTP, amino acids, sugar, lipid, protein and carbohydrate. In certain embodiments, the polyethylene glycol has a molecular weight of between about 6000 and about 10,000, and wherein the salt is selected from the group consisting of sodium chloride, magnesium chloride, calcium chloride, potassium chloride, lithium chloride, barium chloride and cesium chloride. In certain embodiments, the concentration of the polyethylene glycol suitable for binding the nucleic acid to the nanostructure is between about 5% and about 15% and wherein the concentration of salt suitable for binding the nucleic acid to the nanostructure is between about 0.5 M and about 5.0 M. In certain embodiment, the concentration of the polyethylene glycol suitable for binding the nucleic acid to the nanostructure is about 9.375% and the concentration of salt suitable for binding the nucleic acid to the nanostructure is about 0.625 M. In certain embodiment, the concentration of the polyethylene glycol suitable for binding the nucleic acid to the nanostructure is about 10% and the concentration of salt suitable for binding the nucleic acid to the nanostructure is about 2.0 M. In certain embodiment, the concentration of the polyethylene glycol suitable for binding the nucleic acid to the nanostructure is about 13.3% and the concentration of salt suitable for binding the nucleic acid to the nanostructure is about 1.33 M. In certain embodiment, the concentration of the polyethylene glycol suitable for binding the nucleic acid to the nanostructure is about 15% and the concentration of salt suitable for binding the nucleic acid to the nanostructure is about 1.0 M.

In certain embodiments, the kit further comprises a suitable elution buffer, wherein the elution buffer is capable of releasing the bound nucleic acids from the nanostructure into the elution buffer. Examples of suitable elution buffer includes, but not limited to, Tris EDTA solution.

In another aspect, the present disclosure provides a method for reversibly binding at least one nucleic acid through non-hybridization interaction to a nanostructure comprising providing a nanostructure comprising at least one core nanoparticle and a silanization coating on the surface of the core nanoparticle, contacting the nanostructure with a solution containing a first nucleic acid, wherein the concentration of a plurality of ingredients of the solution is adjusted to a concentration suitable for binding the first nucleic acid to the nanostructure, thereby producing a first combination comprising the nanostructure-bound first nucleic acid.

The definitions of “nanostructure”, “nanoparticle,” “silanization coating,” “nucleic acid,” “plurality of ingredients of the solution” have been provided above in the disclosure in connection to the kit.

In certain embodiment, the nucleic acid contained in the solution to be used in the method is at sub-nanogram level.

In certain embodiments, the sub-nanogram level of the nucleic acid is no more than 100 ng, 10 ng, 1 ng or 0.1 ng. For example, the sub-nanogram includes 0.01 ng, 0.02 ng. 0.03 ng, 0.04 ng, 0.05 ng, 0.06 ng, 0.07 ng, 0.08 ng, 0.09 ng, 0.1 ng, 0.2 ng, 0.3 ng, 0.4 ng, 0.5 ng, 0.6 ng, 0.7 ng, 0.8 ng, 0.9 ng, 1.0 ng, or any ranges between any of above mentioned level (e.g., between 0.01 ng and 100 ng, 0.01 ng and 10 ng, 0.01 ng and ing, 0.01 ng and 0.1 ng).

In certain embodiments, the sub-nanogram level of an analyte is no more than 1000 pM, 100 pM, 10 pM, 1 pM, 0.1 pM, 0.01 pM, 0.001 pM (=1 fM) or 0.0001 pM. For example, the sub-nanogram includes 0.001 pM (=1 fM), 0.002 pM. 0.003 pM, 0.004 pM, 0.005 pM, 0.006 pM, 0.007 pM, 0.008 pM, 0.009 pM, 0.01 pM, 0.02 pM, 0.03 pM, 0.04 pM, 0.05 pM, 0.06 pM, 0.07 pM, 0.08 pM g, 0.09 pM, 0.1 pM, 0.1 pM, 0.2 pM, 0.3 pM, 0.4 pM, 0.5 pM, 0.6 pM, 0.7 pM, 0.8 pM, 0.9 pM, 1 pM, 2 pM, 3 pM, 4 pM, 5 pM, 6 pM, 7 pM, 8 pM, 9 pM, 10 pM or any ranges between any of above mentioned level (e.g., between 0.0001 pM and 1000 pM, 0.0001 pM and 100 pM, 0.0001 pM and 10 pM, 0.0001 pM and 1 pM, 0.0001 pM and 0.1 pM, 0.0001 pM and 0.01 pM, 0.0001 pM and 0.001 pM).

In certain embodiments, the method comprises adjusting the concentration of the polyethylene glycol to between about 5% and about 15% and adjusting the concentration of salt to between about 0.5 M and about 5.0 M.

In certain embodiments, the method further comprising: separating the nanostructure from the first combination; contacting the nanostructure separated the first combination with the bound nucleic acid in an elution buffer, whereby the nucleic acid bound to the nanostructure is dissociated from the nanostructure; and separating the nanostructure from the elution buffer.

In certain embodiments, the first nucleic acid has a size of less than 50, 100, 150, 200, 250, or 300 nucleotides.

In certain embodiments, the solution containing the first nucleic acid further comprises a second nucleic acid of smaller size than the first nucleic acid, and wherein the second nucleic acid of smaller size does not bind to the nanostructure at the concentration of the plurality of ingredients suitable for binding the first nucleic acid to the nanostructure, and the method further comprising: separating the nanostructure-bound first nucleic acid from the first combination; permitting the unbound second nucleic acid of smaller size in the first combination to bind to a second nanostructure, producing a second combination comprising nanostructure-bound second nucleic acid of smaller size; separating the nanostructure-bound second nucleic acid of smaller size from the second combination; contacting the nanostructure-bound second nucleic acid of smaller size separated from the second combination with an elution buffer to release the bound second nucleic acid from the second nanostructure into the elution buffer; and separating the second nanostructure from the elution buffer to provide the second nucleic acid that are substantially free of the first nucleic acid.

In certain embodiments, the solution containing the first nucleic acid further comprises a second nucleic acid of smaller size than the first nucleic acid, and wherein the second nucleic acid of smaller size does not bind to the nanostructure at the concentration of the plurality of ingredients suitable for binding the first nucleic acid to the nanostructure, and the method further comprising: separating the nanostructure-bound first nucleic acid from the first combination; permitting the unbound second nucleic acid of smaller size in the first combination to bind to a second nanostructure, producing a second combination comprising nanostructure-bound second nucleic acid of smaller size; separating the nanostructure-bound second nucleic acid of smaller size from the second combination; contacting the nanostructure-bound second nucleic acid of smaller size separated from the second combination with an elution buffer to release the bound second nucleic acid from the second nanostructure into the elution buffer; and separating the second nanostructure from the elution buffer to provide the second nucleic acid that are substantially free of the first nucleic acid.

In yet another aspect, the present disclosure provides a composition for reversibly binding nucleic acids through non-hybridization interaction comprising at least one core nanoparticle, and a silanization coating on the surface of the core nanoparticle, wherein the silanization coating does not include carboxyl group.

In another aspect, the present disclosure provides a method of isolating at least one nucleic acid from a solution containing the nucleic acid comprising combining a nanostructure and the solution, thereby producing a first combination; adjusting the salt concentration and the polyalkylene glycerol concentration of the first combination to concentrations suitable for binding the nucleic acid to the nanostructure, thereby producing a second combination comprising the nucleic acid bound non-specifically to the nanostructure; separating the nanostructure from the second combination; contacting the nanostructure separated with the bound nucleic acid in an elution buffer, whereby the nucleic acid is dissolved in the elution buffer and the nucleic acid bound to the nanostructure is separated from the nanostructure; and separating the nanostructure from the elution buffer.

The term “isolating” nucleic acid refers to the recovery of nucleic acid molecules from a source. While it is not always optimal, the process of recovering nucleic acid may also include recovering some impurities such as protein. It includes, but is not limited to, the physical enrichment of nucleic acid molecules from a source. The term “isolating” may also refer to the duplication or amplification of nucleic acid molecules, without necessarily removing the nucleic acid molecules from the source.

In yet another aspect, the present disclosure provides a method of isolating at least one nucleic acid from a solution containing the nucleic acid comprising combining a nanostructure and the solution, thereby producing a first combination; adjusting the salt concentration and the polyethylene glycerol concentration of the first combination to produce a second combination having a final polyethylene glycol concentration of from about 5% to about 15% and a final sodium chloride concentration of from about 0.5M to about 5.0M, whereby the nucleic acid bound non-specifically to the nanostructure, producing nanostructure having the nucleic acid bound thereto; separating the nanostructure from the second combination; contacting the nanostructure separated with the bound nucleic acid in an elution buffer, whereby the nucleic acid is dissolved in the elution buffer and the nucleic acid bound to the nanostructure is separated from the nanostructure; and separating the nanostructure from the elution buffer.

In yet another aspect, the present disclosure provides a method of separating larger nucleic acids from smaller nucleic acids to obtain larger nucleic acids which are substantially free of the smaller nucleic acids, comprising combining a nanostructure with a solution containing the larger and smaller nucleic acids to produce a first combination; adjusting the salt and polyethylene glycol concentrations of the first combination to concentrations suitable for selectively binding the larger nucleic acids in the solution to the nanostructure, producing a second combination comprising nanostructure-bound larger nucleic acids; separating the nanostructure-bound larger nucleic acids from the second combination; contacting the nanostructure-bound larger nucleic acids separated in c) with an elution buffer to release the bound nucleic acids from the nanostructure into the elution buffer; and separating the nanostructure from the elution buffer to provide nucleic acids that are substantially free of the smaller nucleic acids.

In another aspect, the present disclosure provides a method of separating smaller nucleic acids from larger nucleic acids to obtain the smaller nucleic acids which are substantially free of the larger nucleic acids, comprising combining a first nanostructure with a solution containing the larger and smaller nucleic acids to produce a first combination; adjusting the salt and polyethylene glycol concentrations of the first combination to concentrations suitable for selectively binding the larger nucleic acids in the solution to the nanostructure, producing a second combination comprising nanostructure-bound larger nucleic acids and unbound smaller nucleic acid; separating the nanostructure-bound larger nucleic acids from the second combination; permitting unbound smaller nucleic acids in the second combination to bind to a second nanostructure, producing a third combination comprising nanostructure-bound smaller nucleic acids; separating the nanostructure-bound smaller nucleic acids from the third combination; contacting the nanostructure-bound smaller nucleic acids separated with an elution buffer to release the bound nucleic acids from the second nanostructure into the elution buffer; and separating the second nanostructure from the elution buffer to provide smaller nucleic acids that are substantially free of the larger nucleic acids.

In another aspect, the present disclosure provides a method of separating nucleic acids with medium size range from the smaller or larger nucleic acids to obtain the nucleic acids with medium size range which are substantially free of the smaller and larger nucleic acids, comprising combining a first nanostructure with a solution containing the larger, medium size range and smaller nucleic acids to produce a first combination; adjusting the salt and polyethylene glycol concentrations of the first combination to concentrations suitable for selectively binding the larger nucleic acids in the solution to the nanostructure, producing a second combination comprising nanostructure-bound larger nucleic acids and unbound medium size range and smaller nucleic acid; separating the nanostructure-bound larger nucleic acids from the second combination; permitting unbound medium size range and smaller nucleic acids in the second combination to bind to a second nanostructure, producing a third combination comprising nanostructure-bound medium size range nucleic acids and unbound smaller size nucleic acid; separating the nanostructure-bound medium size range nucleic acids from the third combination; remove the supernatant in the third combination containing the smaller nucleic acid; contacting the nanostructure-bound medium size range nucleic acids separated with an elution buffer to release the bound nucleic acids from the second nanostructure into the elution buffer; and separating the second nanostructure from the elution buffer to provide smaller nucleic acids that are substantially free of the larger nucleic acids.

The following examples are presented to illustrate the present invention. They are not intended to limiting in any manner.

General Methodology

The nanostructure used in the following examples were prepared according to the methods disclosed in this application and those disclosed in US Application Series No US 20100008862 and PCT Application Series No W02013112643A1. The nanostructures were 200-800 nm in diameter. The nanostructure is typically made of nanoparticles having a coating with silocon-containing compounds that contains functional groups. The functional groups on the nanoparticles could include amino, mercapto, carboxyl, phosphonate, biotin, streptavidin, avidin, hydroxyl, alikyl or other hydrophobic molecules, polyethylene glycol or other lydrophilic molecules, and photo cleavable, thermo cleavable or pH responsive linker molecules.

The functional groups can be introduced to the coating compounds during the cross-linking, in particular, during the ending stage of the cross-linking process. The functional groups may also be introduced after the formation of the cross-linking product, for example, by introducing functional groups to the surface of the cross-linking product by chemical modification. In certain embodiments, the functional groups are inherent in the coating.

In general, 20 ng of nanostructure were used per 1000 ng of DNA sample. The nanostructures are stored in 10% PEG 8000, 1M NaCl, 20 mM Tris.Cl storage buffer at a concentration of 2 mg/ml before use. In a typical DNA capture experiment, remove nanostructures from storage and bring them to room temperature. Vortex the nanostructures for 10-20 seconds before use. Remove 20 ng of nanostructures and put into a clean 1.5 ml reaction tube. Collect the nanostructure using magnet and remove the supernatant. Add DNA sample to the nanostructure, vortex or pipette the reaction solution to mix thoroughly. Incubate the nanostructure-DNA reaction at room temperature for 15-30 minutes. After incubation, use the magnet to separate the DNA-bound nanostructure from the solution. Carefully remove the supernatant with a pipette, taking care not to disturb the nanostructure pellet. Keeping the magnet in place, wash the nanostructure pellet by adding 100 ul freshly prepared 70% ethanol. Let stand for 2 minute. Remove and discard the ethanol, repeat the wash step once more. Allow the sample to air dry at room temperature for 5 minutes. Elute the captured DNA from the nanostructures by adding 20 ul of the Elution Buffer, gently pipette to mix well and incubate for 5-10 minute at room temperature, then separate the nanostructures from the eluted DNA with magnet. Transfer the supernatant containing the DNA products to a clean tube. The purified DNA is ready to use for subsequent evaluation.

To evaluate the results of DNA capture, eluted DNA may be resolved in agarose by electrophoresis. All agarose gels were using 1%-3% final agarose (Fisher Scientific) with voltage at 100V-150V and with run time from 40-60 minutes. The gels were post-stained with ethidium bromide and visualized and photographed under UV.

### Example 1

### Isolation of Salmon Sperm DNA Using Nanostructure

Incubate 20 ul of nanostructure or commercially available nanoparticles (Ampure) with 1000 ng salmon sperm DNA for 30 minutes. The bound DNA were eluted in TE (Tris.Cl EDTA) and analyzed on 3% agarose gel. As shown in FIG. 2, the yield of DNA capture using the nanostructure is comparable to the commercially available nanoparticle.

In another experiment, incubate 1000 ng genomic DNA of OC1-LY8 cells and salmon sperm DNA with 20 ul of nanostructure or commercially available nanoparticles (Ampure). DNA captured by nanostructure were eluted and quantified using Picogreen fluorescent reagent (Life Technologies). As shown in FIG. 3, the capturing efficiency of nanostructure and commercially available nanoparticles are comparable.

In yet another experiment, incubate 5,000 ng or 10,000 ng of salmon sperm DNA with 20 ul nanostructure or commercially available nanoparticles (Ampure) under room temperature for 1 hour, then eluted and analyzed on 3% agrose gel. As shown in FIG. 4, using nanostructure yield a significant higher binding capacity than commercially available nanoparticles under the saturation conditions.

### Example 2

### The Recovery Rate of PCR Product Clean Up Using Nanostructure

Remove 20 ul of nanostructures and put into a clean 1.5 ml reaction tube. Collect the nanostructure using magnet and remove the supernatant. Add 100, 250, 500 or 1,000 ng DNA ladder to the nanostructure, vortex or pipette the reaction solution to mix thoroughly. Incubate the nanostructure-DNA reaction at room temperature for 30 minutes. After incubation, use the magnet to separate the DNA-bound nanostructure from the solution. Carefully remove the supernatant with a pipette, taking care not to disturb the nanostructure pellet. Keeping the magnet in place, wash the nanostructure pellet by adding 100 ul freshly prepared 70% ethanol. Let stand for 2 minute. Remove and discard the ethanol, repeat the wash step once more. Allow the sample to air dry at room temperature for 5 minutes. Elute the captured DNA from the nanostructures by adding 20 ul of the Elution Buffer, gently pipette to mix well and incubate for 5-10 minute at room temperature, then separate the nanostructures from the eluted DNA with magnet. Transfer the supernatant containing the DNA products to a clean tube. The purified DNA is quantified using Picogreen fluorescent reagent. As shown in FIG. 6, the recovery rate is above 75% percent and remains stable within all DNA concentration range.

### Example 3

### Isolation of Trace Amount of DNA Using Nanostructure

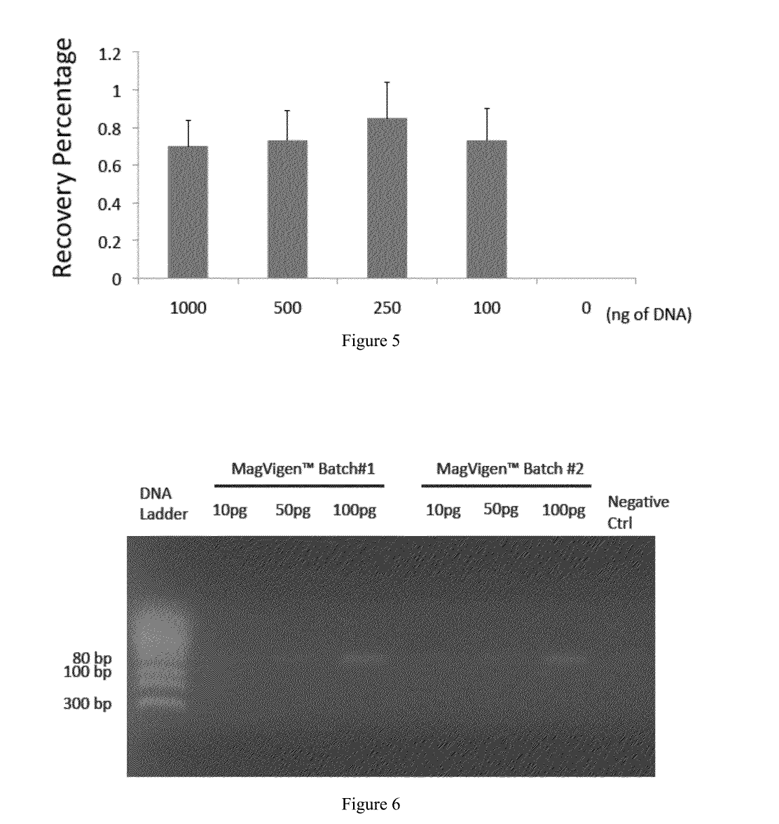

A DNA template of 82 bp was spiked into 1 ml of human blood sample, a biotin-labeled primer was used to specifically target this 82 bp template in blood sample. The blood sample was boiled for 10 minutes to denature DNA, then 10 pmol biotin-labeled primer and 40 ul nanostructure coated with Streptavidin were mixed with the blood sample at room temperature for 1 hour. The DNA template was specifically captured to the nanostructure by Streptavidine-biotin complex. The eluted DNA was further amplified by PCR and analyzed on 3% agrose gel. FIG. 5 shows that Streptavidin coated nanostructure can detect DNA to as low as 50 pg/ml blood, and there is no-unspecific binding.

### Example 4

### Isolation of Small Size DNA Using Nanostructure

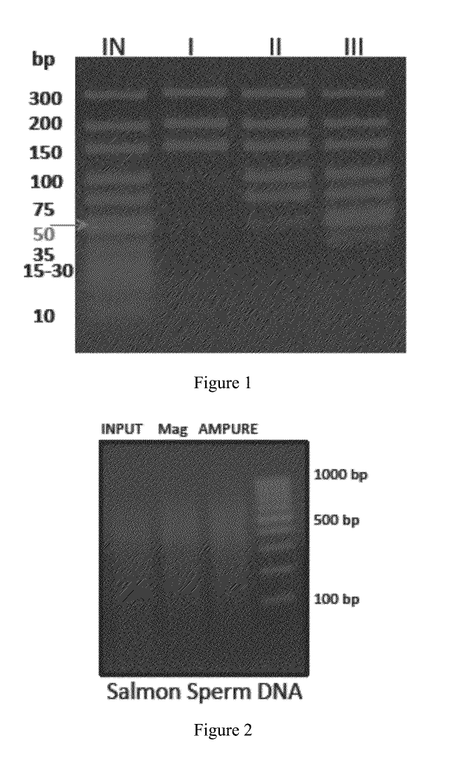

Remove 20 ul of nanostructures and put into a clean 1.5 ml reaction tube. Collect the nanostructure using magnet and remove the supernatant. Resuspend the nanostructure in 40 ul capture buffer. Add 1,000 ng Ultra Low DNA ladder (Fisher Scientific) to the nanostructure, vortex or pipette the reaction solution to mix thoroughly. Incubate the nanostructure-DNA reaction at room temperature for 30 minutes. After incubation, use the magnet to separate the DNA-bound nanostructure from the solution. Carefully remove the supernatant with a pipette, taking care not to disturb the nanostructure pellet. Keeping the magnet in place, wash the nanostructure pellet by adding 100 ul freshly prepared 70% ethanol. Let stand for 2 minute. Remove and discard the ethanol, repeat the wash step once more. Allow the sample to air dry at room temperature for 5 minutes. Elute the captured DNA from the nanostructures by adding 20 ul of the Elution Buffer, gently pipette to mix well and incubate for 5-10 minute at room temperature, then separate the nanostructures from the eluted DNA with magnet. Transfer the supernatant containing the DNA products to a clean tube. The purified DNA is resolved on 3% agarose. As shown in FIG. 1, using nanostucture together with proper buffer was capable of capturing DNA molecular size to as small as 150 bp/75 bp/50 bp.

### Example 5

### Isolation DNA from Plasma Using Nanostructure

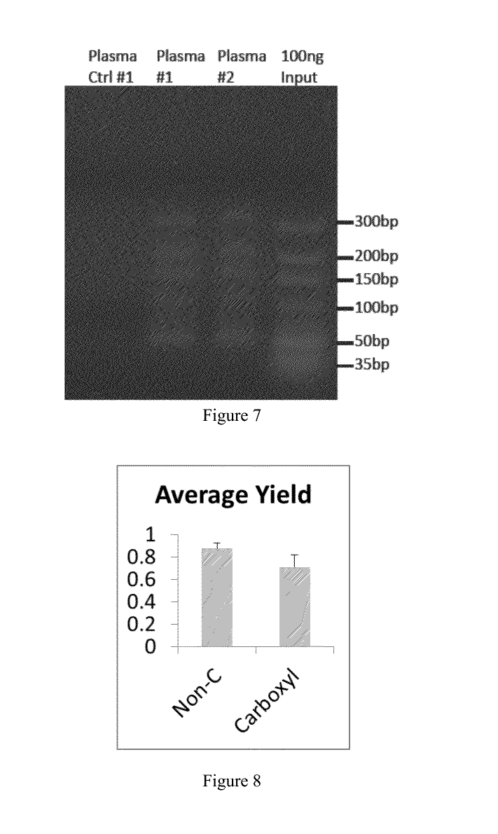

Combine 120 ul plasma (Zen-Bio) with 120 ul plasma digest buffer 20 mM Tris.Cl, 700 U/ml proteinase K, 0.1 mM hydrochloride, 5% Glucose, 2 mM EDTA. Add 2.5 ul proteinase K (New England Lab) for 60 min at 37 degree. Add 400 ul plasma DNA binding solution (25% PEG 8000, 2.5M NaCl, 20 mM Tris.Cl, 10% Glucose). Incubate at RT for 60 mins. Nanoparticles were pelleted on a magnet stand and washed with 400 ul freshly-made 80% ethanol twice. Pellets were air-dry for 5 minutes and bounded DNA were then eluted from nanostructures in 50 ul Elution buffer and analyzed on 3% Agrose gel. The results were shown in FIG. 7.

### Example 6

### Comparison Between Non-Carboxyl Coated and Carboxyl Coated Nanostructure

Remove 20 ul of non-carboxyl coated or carboxyl coated nanostructures and put into a clean 1.5 ml reaction tube. Collect the nanostructure using magnet and remove the supernatant. Resuspend the nanostructure in 40 ul capture buffer. The capture buffer contains 25% PEG 8,000, 2.5 M NaCl, and 20 mM Tris.Cl (pH7.2). Add 1,000 ng of 100˜10,000 bp DNA ladder (Fisher Scientific) to the nanostructure, vortex or pipette the reaction solution to mix thoroughly. Incubate the nanostructure-DNA reaction at room temperature for 30 minutes. After incubation, use the magnet to separate the DNA-bound nanostructure from the solution. Carefully remove the supernatant with a pipette, taking care not to disturb the nanostructure pellet. Keeping the magnet in place, wash the nanostructure pellet by adding 100 ul freshly prepared 70% ethanol. Let stand for 2 minute. Remove and discard the ethanol, repeat the wash step once more. Allow the sample to air dry at room temperature for 5 minutes. Elute the captured DNA from the nanostructures by adding 20 ul of the Elution Buffer, gently pipette to mix well and incubate for 5-10 minute at room temperature, then separate the nanostructures from the eluted DNA with magnet. Transfer the supernatant containing the DNA products to a clean tube. The purified DNA is quantified using Picogreen fluorescent reagent. As shown in FIG. 8, the yield of non-Carboxyl coated nanostructure is higher than the Carboxyl coated nanostructure.

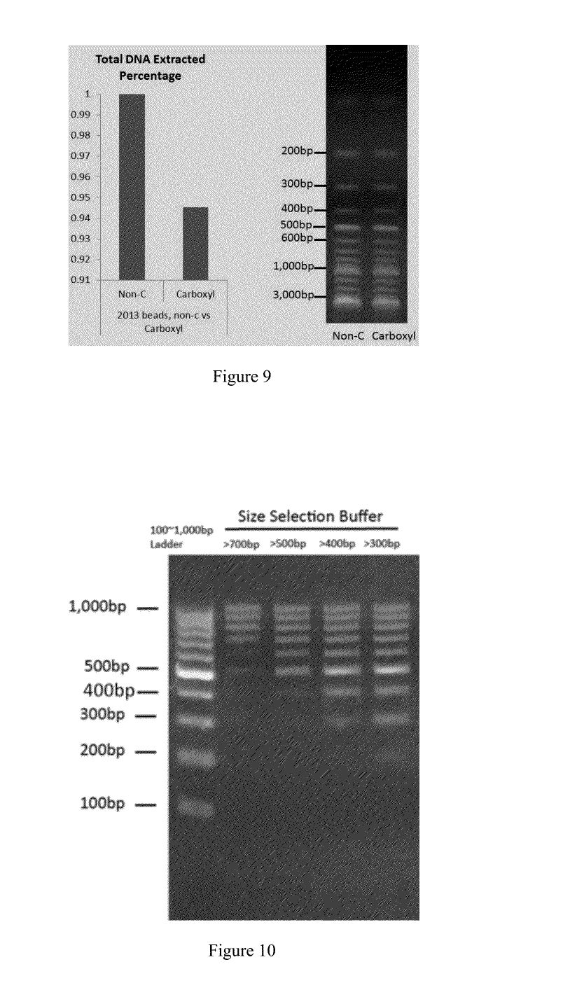

In another experiment, DNA ladder were isolated using non-carboxyl coated or carboxyl coated nanostructure with size selection buffer for DNA having size>200 bp. The bounded DNA were eluted and resolved on 3% agarose or quantified using Pecogreen fluorescent reagent. As shown if FIG. 9, the yield of non-Carboxyl coated nanostructure is higher than the Carboxyl coated nanostructure.

### Example 7

### Selective Isolation of Nucleic Acid Using Nanostructure

Incubate 1000 ng 100˜1,000 bp DNA ladder (Fisher Scientific) with 20 ug nanostructure in 20 ul DNA size selection buffers for 30 minutes. The concentration of salt and PEG of different size selection buffer is as following:

Size selection buffer for DNA>700 bp: 9.375% PEG 8000, 0.625M NaCl, 20 mM Tris.Cl. (pH7.2)

Size selection buffer for DNA>500 bp: 10% PEG 8000, 2.0M NaCl, 20 mM Tris.Cl. (pH 7.2)

Size selection buffer for DNA>400 bp: 13.3% PEG 8000, 1.33M NaCl, 20 mM Tris.Cl. (pH 7.2)

Size selection buffer for DNA>300 bp: 15% PEG 8000, 1.0M NaCl, 20 mM Tris.Cl. (pH 7.2)

1,000 ng DNA Ladder of 100˜1,000 bp (Fisher Scientific) were mixed with 20 ul Magvigen DNA Size-Selection nanoparticles in dedicated Capture Buffer-700/500/400/300 at room temperature for 30 minutes, respectively. The nanoparticles were pelleted on magnet stand and washed with 100 ul fresh 70% ethanol twice. The captured ladder were eluted and analyzed on 3% agrose gel. Each Capture Buffer was capable of capturing DNA molecular size to as low as 700 bp/500 bp/400 bp/300 bp. As illustrated in FIG. 10, using different size selection buffer resulted in selective binding of DNA of different sizes to the nanostructure.

### Example 8

### FIG. 9 Isolation of a Medium Size Range of DNA Using Nanostructure

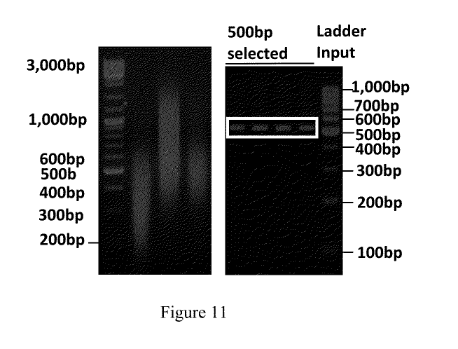

Remove 80 ul nanostructure form storage and bring them to room temperature. Vortex nanostructure for 10-20 seconds before use. Pellet down the nanostructure using a magnet and remove the supernatant. Resuspend the nanostructure in 60 ul capture buffer (9.375% PEG 8000, 0.625M NaCl, 20 mM Tris.Cl. (pH7.2)). Add 40 ul 5,000 ng of salmon sperm DNA (Life Technologies) or 1,000 ng DNA Ladder od 100˜1,000 bp (Fisher Scientific) to the nanostructure at room temperature for 60 minutes. The un-captured DNA was separated from nanoparticle pellet on a magnetic stand. The supernatant was transferred to a new tube containing 160 ng nanostructures and 20 ul capture buffer (15% PEG 8000, 1.0M NaCl, 20 mM Tris.Cl. (pH 7.2)). The mixture were vortex thoroughly and incubated at RT for another 60 minutes. The captured DNA were washed twice with fresh 70% ethanol and eluted and analyzed on 3% agarose gel. FIG. 11 illustrates that the DNA having size from 300 bp to 600 bp were isolated.

While the invention has been particularly shown and described with reference to specific embodiments (some of which are preferred embodiments), it should be understood by those having skill in the art that various changes in form and detail may be made therein without departing from the spirit and scope of the present invention as disclosed herein.

What is claimed is:

1) A kit comprising:
(a) a nanostructure comprising
(i) at least one core nanoparticle, and
(ii) a silanization coating on the surface of the core nanoparticle; and
(b) a binding buffer comprising a plurality of ingredients at concentration suitable to adjust the concentration of the plurality of ingredients in a solution containing at least one nucleic acid to concentration suitable for binding the nucleic acid through non-hybridization interaction to the nanostructure.

2) The kit of claim 1, wherein the silanization coating does not include a carboxyl group.

3) The kit of claim 1, wherein the core nanoparticle comprises a superparramagnetic iron oxide (SPIO) nanoparticle.

4) The kit of claim 1, wherein the silanization coating forms a low density, porous 3-D structure.

5) The kit of claim 1, wherein the plurality of ingredients of the solution comprises salt and polyethylene glycol.

6) The kit of claim 5, wherein the plurality of ingredients of the solution further comprises ingredients selected from the group consisting of acid, base, dNTP, amino acids, sugar, lipid, protein and carbohydrate.

7) The kit of claim 5, wherein the polyethylene glycol has a molecular weight of between about 6000 and about 10,000, and wherein the salt is selected from the group consisting of sodium chloride, magnesium chloride, calcium chloride, potassium chloride, lithium chloride, barium chloride and cesium chloride.

8) The kit of claim 7, wherein the concentration of the polyethylene glycol suitable for binding the nucleic acid to the nanostructure is between about 5% and about 15% and wherein the concentration of salt suitable for binding the nucleic acid to the nanostructure is between about 0.5 M and about 5.0 M.

9) The kit of claim 7, wherein the concentration of the polyethylene glycol suitable for binding the nucleic acid to the nanostructure is about 9.375% and the concentration of salt suitable for binding the nucleic acid to the nanostructure is about 0.625 M.

10) The kit of claim 7, wherein the concentration of the polyethylene glycol suitable for binding the nucleic acid to the nanostructure is about 10% and the concentration of salt suitable for binding the nucleic acid to the nanostructure is about 2.0 M.

11) The kit of claim 7, wherein the concentration of the polyethylene glycol suitable for binding the nucleic acid to the nanostructure is about 13.3% and the concentration of salt suitable for binding the nucleic acid to the nanostructure is about 1.33 M.

12) The kit of claim 7, wherein the concentration of the polyethylene glycol suitable for binding the nucleic acid to the nanostructure is about 15% and the concentration of salt suitable for binding the nucleic acid to the nanostructure is about 1.0 M.

13) The kit of claim 1, further comprising a suitable elution buffer, wherein the elution buffer is capable of releasing the bound nucleic acids from the nanostructure into the elution buffer.

14) A method for reversibly binding at least one nucleic acid through non-hybridization interaction to a nanostructure comprising:
(a) providing a nanostructure comprising at least one core nanoparticle and a silanization coating on the surface of the core nanoparticle;
(b) contacting the nanostructure with a solution containing a first nucleic acid;
wherein the concentration of a plurality of ingredients of the solution is adjusted to a concentration suitable for binding the first nucleic acid to the nanostructure;
thereby producing a first combination comprising the nanostructure-bound first nucleic acid.

15) The method of claim 14, wherein the nucleic acid contained in the solution is at sub-nanogram level.

16) The method of claim 14, wherein the plurality of ingredients of the solution comprises salt and polyethylene glycol.

17) The method of claim 14, wherein the solution containing the first nucleic acid is a biological sample.

18) The method of claim 14, further comprising:
(c) separating the nanostructure from the first combination;
(d) contacting the nanostructure separated the first combination with the bound nucleic acid in an elution buffer, whereby the nucleic acid bound to the nanostructure is dissociated from the nanostructure; and
(e) separating the nanostructure from the elution buffer.

19) The method of claim 14, wherein the solution containing the first nucleic acid further comprises a second nucleic acid of smaller size than the first nucleic acid, and wherein the second nucleic acid of smaller size does not bind to the nanostructure at the concentration of the plurality of ingredients suitable for binding the first nucleic acid to the nanostructure, further comprising:
(c) separating the nanostructure-bound first nucleic acid from the first combination;
(d) permitting the unbound second nucleic acid of smaller size in the first combination to bind to a second nanostructure, producing a second combination comprising nanostructure-bound second nucleic acid of smaller size;
(e) separating the nanostructure-bound second nucleic acid of smaller size from the second combination;
(f) contacting the nanostructure-bound second nucleic acid of smaller size separated in e) with an elution buffer to release the bound second nucleic acid from the second nanostructure into the elution buffer; and
(g) separating the second nanostructure from the elution buffer to provide the second nucleic acid that are substantially free of the first nucleic acid.

20) The method of claim 14, wherein the solution containing the first nucleic acid further comprises a second nucleic acid of smaller size than the first nucleic acid, and wherein the second nucleic acid of smaller size does not bind to the nanostructure at the concentration of the plurality of ingredients suitable for binding the first nucleic acid to the nanostructure, further comprising:
(c) separating the nanostructure-bound first nucleic acid from the first combination;
(d) permitting the unbound second nucleic acid of smaller size in the first combination to bind to a second nanostructure, producing a second combination comprising nanostructure-bound second nucleic acid of smaller size;
(e) separating the nanostructure-bound second nucleic acid of smaller size from the second combination;
(f) contacting the nanostructure-bound second nucleic acid of smaller size separated in e) with an elution buffer to release the bound second nucleic acid from the second nanostructure into the elution buffer; and
(g) separating the second nanostructure from the elution buffer to provide the second nucleic acid that are substantially free of the first nucleic acid.

21) A composition for reversibly binding nucleic acids through non-hybridization interaction comprising:
(a) at least one core nanoparticle, and
(b) a silanization coating on the surface of the core nanoparticle, wherein the silanization coating does not include carboxyl group.
